(19) United States
(12) Patent Application Publication
(10) Pub. No.: US 2021/0196781 A1
(43) Pub. Date: Jul. 1, 2021

(54) SKIN BALM

(71) Applicant: Faye Williams, Jamaica, NY (US)

(72) Inventor: Faye Williams, Jamaica, NY (US)

(21) Appl. No.: 17/247,882

(22) Filed: Dec. 29, 2020

Related U.S. Application Data
(60) Provisional application No. 62/954,881, filed on Dec. 30, 2019.

(51) Int. Cl. A61K 36/889; A61K 8/92; A61K 8/9794; A61K 8/9789; A61Q 19/08; A61K 8/67; A61K 31/07; A61K 35/644; A61K 36/736; A61K 36/36; A61Q 17/04

(52) U.S. Cl. CPC A61K 36/889; A61K 8/927; A61K 8/9794; A61K 8/9789; A61K 8/922; A61Q 19/08; A61K 9/0014; A61K 31/07; A61K 35/644; A61K 36/736; A61K 36/36; A61Q 17/04; A61K 8/671

(57) ABSTRACT

An antioxidant and anti-inflammatory composition includes coconut oil, jojoba oil, argan butter, beeswax, and an essential oil blend. a method of therapeutically treating a malady includes applying an amount of the antioxidant and anti-inflammatory composition on a subject's skin. A method of manufacturing an antioxidant and anti-inflammatory skin balm includes melting beeswax; combining coconut oil, argan butter, almond butter, and jojoba oil with the melted beeswax; stirring to form a melted blend; adding a blend of essential oils; and cooling the melted blend. The composition is useful for muscle ache, muscle soreness, joint discomfort, cellulite, obesity, edema, poor circulation, headache, heart disease, hypertension, tendonitis, cramps, inflammation, eczema, psoriasis, gout, ulcers, arthritis, rheumatism, muscle spasms, acne, chapped skin, irritated skin, localized pain, and dry hair.

### CROSS-REFERENCE TO RELATED APPLICATION

This application claims the benefit of priority of U.S. provisional application No. 62/954,881, filed Dec. 30, 2019, the contents of which are herein incorporated by reference.

### BACKGROUND OF THE INVENTION

The present invention relates to a skin balm and, more particularly, to an antibacterial composition effective to relieve pain and inflammation.

Individuals suffering from more than one malady such as muscle aches and soreness, loss of skin elasticity, and hair loss, as well as having a desire for focused weight loss, must use multiple products to address their needs. Commercially available products have one or two of anti-bacterial, anti-inflammatory, and pain-reliever functions but not all three.

As can be seen, there is a need for a product with multiple applications, reducing the need to buy separate products.

The present invention provides a multi-purpose skin cream, having anti-bacterial, anti-inflammatory, and pain-relieving properties.

The multi-purpose skin cream improves skin elasticity to allow it to stay firm. It supports focused weight loss by producing a thermogenic response for increased perspiration. The inventive cream relieves muscle ache, soreness, and pains related to physical activity. The composition stimulates hair growth and improves circulation due to the combination of natural compounds.

### SUMMARY OF THE INVENTION

In one aspect of the present invention, an antioxidant and anti-inflammatory composition is provided comprising coconut oil, jojoba oil, argan butter, beeswax, and an essential oil blend.

In another aspect of the present invention, a method of therapeutic treatment of a malady, is provided, comprising topically applying an effective amount of the antioxidant and anti-inflammatory composition on a subject's skin, wherein the malady is selected from the group consisting of: muscle ache, muscle soreness, joint discomfort, cellulite, obesity, edema, poor circulation, headache, heart disease, hypertension, tendonitis, cramps, inflammation, eczema, psoriasis, gout, ulcers, arthritis, rheumatism, muscle spasms, acne, chapped skin, irritated skin, localized pain, and dry hair.

In another aspect of the present invention, a method of manufacturing an antioxidant and anti-inflammatory skin balm is provided, comprising melting beeswax to form a melted beeswax; combining coconut oil, argan butter, almond butter, and jojoba oil with the melted beeswax; stirring the coconut oil, argan butter, almond butter, jojoba oil, and the melted beeswax over heat until melted and blended to form a melted blend; adding a blend of essential oils to the melted blend; and cooling the melted blend.

These and other features, aspects and advantages of the present invention will become better understood with reference to the following drawings, description, and claims.

### BRIEF DESCRIPTION OF THE DRAWINGS

FIG. 1 is a chart illustrating ingredients of a composition according to an embodiment of the present invention;

FIG. 2A is a schematic showing a first step of a production process thereof;

FIG. 2B is a schematic showing a second step thereof; and

FIG. 2C is a schematic showing a third step thereof.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

As used herein, a composition having a component present in a % by volume has a total composition of 100% by volume.

The transitional phrase “consisting essentially of” limits the scope of a claim to the specified materials or steps “and those that do not materially affect the basic and novel characteristic(s)” of the claimed invention. With respect to product claims, “consisting essentially of” and like terminology refers to the recited components and excludes other ingredients which would substantially change the basic and novel characteristics of the composition or article. Unless otherwise indicated or readily apparent, a composition or article consists essentially of the recited components when the composition or article includes 90% or more by weight of the recited components. That is, the terminology excludes more than 10% unrecited components.

As used herein, the term “thermogenic” refers to cooling, soothing, menthol effects experienced when a component is applied and/or massaged into skin.

Broadly, one embodiment of the present invention is an antioxidant and anti-inflammatory composition for topical application comprising coconut oil, jojoba oil, argan butter, beeswax, and an essential oil blend.

Generally, all the components are 100% natural materials. Generally, all components are Vegan Certified. All components are believed to be hypo-allergenic if not ingested.

Coconut oil has been used for hair grooming and has been found to reduce protein loss in hair. Massage with coconut oil is believed to help increase blood circulation. Preferably, the coconut oil is 100% organic.

Pure jojoba oil and jojoba butter are believed to support blood circulation and may be used on skin, hair, or cuticles.

Argan oil and argan butter are believed to have antioxidant and anti-Inflammatory properties and are believed to promote skin elasticity.

Beeswax is believed to have antibacterial properties and serves as a barrier, retaining moisture in the skin.

An essential oil blend may comprise at least two oils, at least one of which is selected from the group consisting of clove oil, peppermint oil, wintergreen oil, and helichrysum oil.

Clove oil is believed to have antibacterial and thermogenic properties and to be effective as a mosquito repellent and an analgesic.

Peppermint oil is believed to have antibacterial and thermogenic properties and to be effective as an anti-Inflammatory. Peppermint oil may be used to treat arthritis and rheumatism.

Wintergreen is believed to have thermogenic properties.

Helichrysum is believed to be effective as an analgesic, an antispasmodic, an antibacterial agent, and an anti-inflammatory agent useful to relieve muscle spasms, inflammation, and pain.

In some embodiments, the composition further comprises almond oil. Almond oil is believed to protect skin from ultraviolet (UV) radiation damage, as well as to promote healing of chapped and irritated skin.

In some embodiments, the composition further comprises Vitamin A. Vitamin A, which includes fat-soluble compounds such as retinol, may be used to reduce acne.

In some embodiments, the inventive composition comprises an analgesic such as clove oil and/or helichrysum. In some cases, other naturally occurring (botanically derived) analgesic components may be used in addition to, or instead of, clove oil or helichrysum.

Coconut oil may be present in an amount of about 53% to about 61% by volume. The composition may have one or more ratio selected from the group consisting of: a ratio of coconut oil:argan butter of about 4:1, a ratio of coconut oil:jojoba oil of about 4:1, a ratio of coconut oil:almond oil of about 4:1, a ratio of coconut oil:beeswax of about 4:1, and a ratio of coconut oil:essential oils of about 118:1.

Argan butter may be present in an amount of about 13% to about 15% by volume. The composition may have one or more ratio selected from the group consisting of: a ratio of argan butter:jojoba oil of about 1:1, a ratio of argan butter:almond oil of about 1:1, a ratio of argan oil to beeswax of about 2:1, and a ratio of argan oil:essential oils of about 30:1.

Jojoba oil may be present in an amount of about 13% to about 15% by volume. The composition may have one or more ratio selected from the group consisting of: a ratio of jojoba oil to almond oil of about 1:1, a ratio of jojoba oil:beeswax of about 2:1, and a ratio of jojoba oil:essential oils of about 30:1.

Almond oil may be present in an amount of about 13% to about 15% by volume. The composition may have one or more ratio selected from the group consisting of: a ratio of almond oil:beeswax of about 2:1 and a ratio of almond oil:essential oils of about 30:1.

Beeswax may be present in an amount of about 6% to about 8% by volume. The composition may have a ratio of beeswax:essential oils of about 15:1.

A blend of essential oils may be present in an amount of about 0.004% to about 0.005% by volume.

The inventive composition may be used in an effective amount as a remedy for a variety of maladies, including muscle and joint discomfort (including muscle ache or soreness), cellulite, obesity, edema, poor circulation, headache, heart disease, hypertension, tendonitis, cramps, inflammation, eczema, hair care, psoriasis, gout, and ulcers. Therapeutic treatment may include topical application of the inventive skin balm on a subject's skin.

The inventive composition may be used for hair grooming to reduce protein loss in hair. The composition may function as a hair, skin, and cuticle conditioner. With antioxidant and anti-inflammatory properties, the skin balm may be effective to treat arthritis and rheumatism.

In some embodiments, the composition is believed to be effective as a mosquito repellent, as an antispasmodic, to protect skin from UV radiation damage, to reduce bodily acne. Furthermore, the composition is believed to promote healing of chapped and irritated skin.

The inventive composition may be used according to a variety of methods to achieve various results, as discussed below.

To achieve increased sweat production, the inventive skin balm may be applied to any areas of the body before a workout or sauna. Application to the face is not recommended for this purpose. The inventive balm may also be used under a waist trainer and may help achieve weight loss goals.

The inventive balm may be applied liberally to the body in conjunction with a body massage.

The inventive composition may be used for localized pain relief by applying a predetermined amount to areas of discomfort and massaging the composition into the skin.

The balm of the present invention may be used on the scalp to stimulate the scalp and may be applied to dry hair daily. Preferably, the scalp is clean prior to application. The user may part hair and apply a predetermined amount to the scalp with fingertips. The user may then massage the composition into the scalp and hair and may style the hair as usual.

The skin balm composition may be applied after a shower as a moisturizer, particularly during the winter season. The user may apply an amount of balm onto damp skin and may rub the composition into the skin. The user may use a towel to pat dry and remove any excess composition.

The inventive balm may be manufactured by a method including melting the botanical butters on low heat, adding beeswax, letting the mixture cool slightly, and adding essential oils. The composition may be poured into a sterile container.

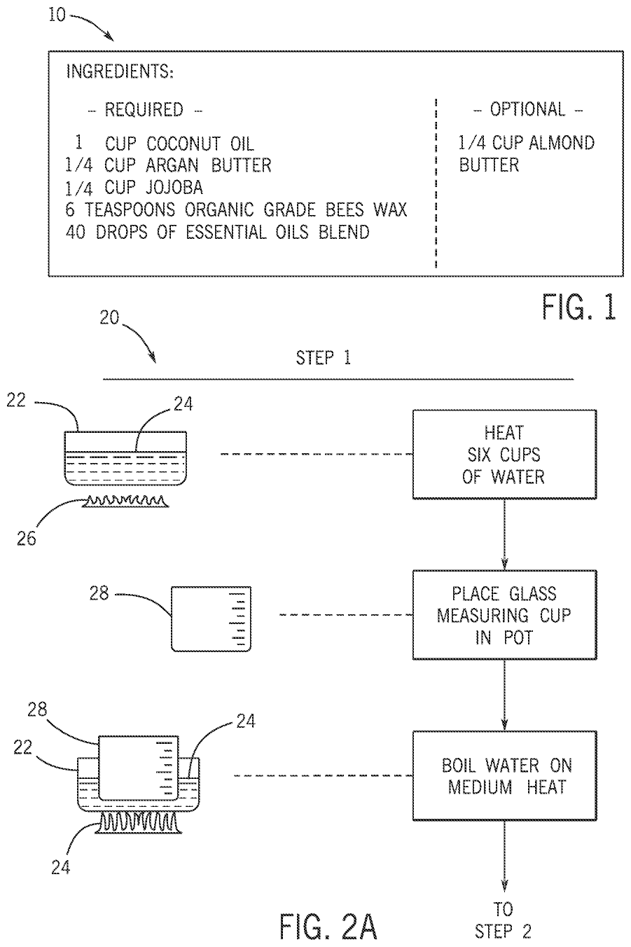

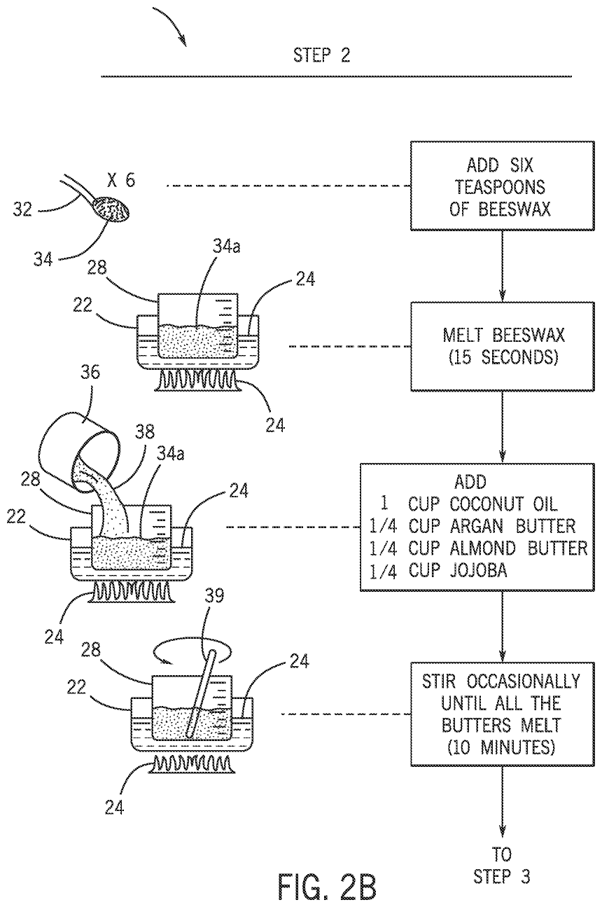

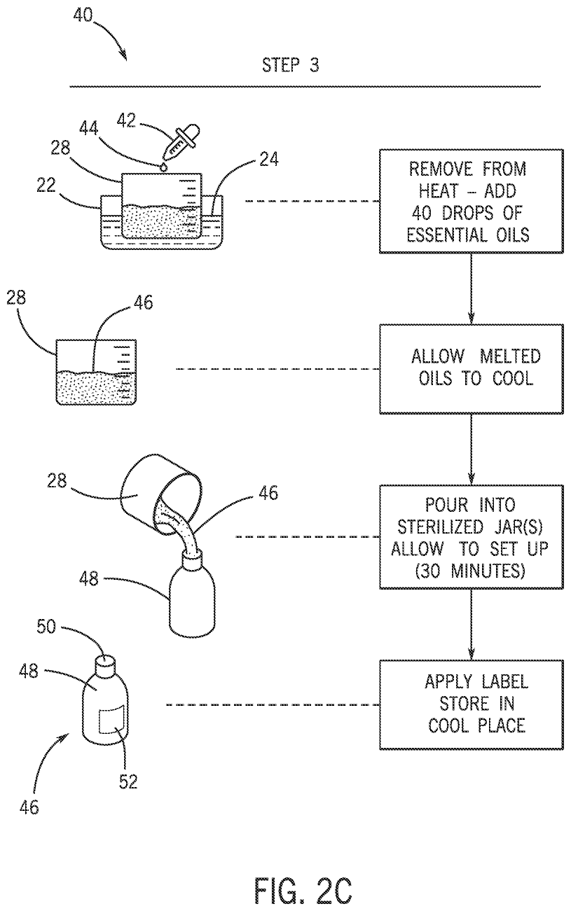

Referring to FIGS. 1 and 2A-2C, FIG. 1 illustrates relative amounts of required ingredients 10 according to an embodiment of the present invention, including coconut oil, argan butter, jojoba oil, beeswax, and a blend of essential oils. In some cases, Almond butter may also be added.

FIGS. 2A, 2B, and 2C illustrate an example of a production process of the inventive composition. The first step 20 includes heating water 24 in a pan 22 over a heat source 26, placing a measuring cup 28 in the water, and boiling the water on medium heat. The second step 30 includes measuring beeswax 34 with a teaspoon 32 and adding the beeswax to the heated measuring cup 28 to produce melted beeswax 34a. Additional ingredients 38, including coconut oil, argan butter, almond butter, and jojoba, may be mixed in an ingredient container 36 and added to the heated measuring cup 28. The additional ingredients 38 may then be mixed into the melted beeswax 34a with a stirrer 39. The melted mixture in the measuring cup 28 is removed from the heat in the third step 40. Using an eye dropper 42, essential oils 44 may be added before the mixture is cool. Once the product 46 has cooled, it may be poured into a bottle 48. The bottle 48 may be capped 50 and labeled 52.

It should be understood, of course, that the foregoing relates to exemplary embodiments of the invention and that modifications may be made without departing from the spirit and scope of the invention as set forth in the following claims.

What is claimed is:

1. An antioxidant and anti-inflammatory composition comprising coconut oil, jojoba oil, argan butter, beeswax, and an essential oil blend.

2. The antioxidant and anti-inflammatory composition of claim 1, wherein the essential oil blend comprises at least one oil selected from the group consisting of: clove oil, peppermint oil, wintergreen oil, and helichrysum oil.

3. The antioxidant and anti-inflammatory composition of claim 1, wherein the essential oil blend comprises clove oil, peppermint oil, and wintergreen oil.

4. The antioxidant and anti-inflammatory composition of claim 1, further comprising almond oil.

5. The antioxidant and anti-inflammatory composition of claim 1, further comprising vitamin A.

6. The antioxidant and anti-inflammatory composition of claim 1, further comprising a botanically derived analgesic.

7. The antioxidant and anti-inflammatory composition of claim 1, wherein coconut oil is present in an amount of about 53% to about 61% by volume.

8. The antioxidant and anti-inflammatory composition of claim 1, wherein argan butter is present in an amount of about 13% to about 15% by volume.

9. The antioxidant and anti-inflammatory composition of claim 1, wherein jojoba oil is present in an amount of about 13% to about 15% by volume.

10. The antioxidant and anti-inflammatory composition of claim 1, wherein beeswax is present in an amount of about 6% to about 8% by volume.

11. A method of therapeutic treatment of a malady, comprising:
topically applying an effective amount of the antioxidant and anti-inflammatory composition of claim 1 on a subject's skin, wherein the malady is selected from the group consisting of: muscle ache, muscle soreness, joint discomfort, cellulite, obesity, edema, poor circulation, headache, heart disease, hypertension, tendonitis, cramps, inflammation, eczema, psoriasis, gout, ulcers, arthritis, rheumatism, muscle spasms, acne, chapped skin, irritated skin, localized pain, and dry hair.

12. A method of manufacturing an antioxidant and anti-inflammatory skin balm, comprising:
a. melting beeswax to form a melted beeswax;
b. combining coconut oil, argan butter, almond butter, and jojoba oil with the melted beeswax;
c. stirring the coconut oil, argan butter, almond butter, jojoba oil, and the melted beeswax over heat until melted and blended to form a melted blend;
d. adding a blend of essential oils to the melted blend; and
e. cooling the melted blend.
